(19) United States
(12) Patent Application Publication
(10) Pub. No.: US 2008/0112989 A1
(43) Pub. Date: May 15, 2008

(54) Encapsulation method

(75) Inventors: Robert B. Wieland, Cincinnati, OH (US); Jon C. Soper, Union, KY (US)

Correspondence Address: CURATOLO SIDOTI CO., LPA, 24500 CENTER RIDGE ROAD, SUITE 280, CLEVELAND, OH, 44145, US

(73) Assignee: Givaudan S.A., Vernier (CH)

(21) Appl. No.: 11/904,183

(22) Filed: Sep. 26, 2007

Related U.S. Application Data
(63) Continuation-in-part of application No. PCT/CH2006/000253, filed on May 10, 2006.
(60) Provisional application No. 60/679,861, filed on May 11, 2005.

(51) Int. Cl. A61K 9/48

(52) U.S. Cl. 424/401; 424/451; 264/4.1

(57) ABSTRACT

A method of loading liquid lipophilic compounds to capsules having a hydrogel shell and an oil core, wherein said compound, said capsules, and water and a solvent that is miscible with both water and lipophilic compound are thoroughly mixed and incubated for a sufficient time to provide capsules loaded with a lipophilic flavor or fragrance compound. The capsules are useful for providing flavors or fragrances to a wide variety of products such as foodstuffs, beverages, medicines and tobacco products.

### CROSS REFERENCE TO RELATED APPLICATIONS

This application is a continuation-in-part of International Application No. PCT/CH2006/000253 filed 10 May 2006, under 35 USC §120 and §365(c), which claims the benefit of the filing date of U.S. Provisional Application No. 60/679,681 filed 11 May 2005.

### TECHNICAL FIELD

The invention relates to a method of encapsulating liquid lipophilic compounds, particularly flavor or fragrance compounds, and to resulting loaded capsules.

### BACKGROUND

Liquid lipophilic compounds (“lipophiles”) include flavor or fragrance compounds such as natural oils and lipophilic compounds of plants or part of plants (such as leaves or fruits), in particular limonene, orange oil, or lemon oil. They also include other materials such as pharmaceuticals and “nutraceuticals”. All such materials are partly water-soluble.

Methods for loading amphiphilic compounds are known to the art. For example, one method uses water to load flavors and fragrances to capsules with hydrogel shells. It involves the addition of flavor compounds in the presence of water to blank capsules. The water allows the transport of the flavor compounds through the water-containing hydrogel shell into the oil core by aqueous diffusion according to its partition coefficient equilibrium. However, when loading lipophiles, the transport to the core of the capsule through the water swollen wall is not very efficient, leading to a less effective encapsulation of these substances.

### SUMMARY

A method of loading a liquid lipophilic compound to capsules having a hydrogel shell and an oil core is provided, wherein said lipophilic compound, said capsules, and water and a solvent that is miscible with both water and the lipophilic compound are thoroughly mixed and incubated for a sufficient time to provide capsules loaded with the lipophilic compound.

### DETAILED DESCRIPTION

A method of encapsulating liquid lipophilic compounds is provided, particularly flavor or fragrance compounds, and to the resulting loaded capsules.

It has now been found that that the loading efficiency of such materials can be improved by using a particular method of loading. The method comprises loading a liquid lipophilic compound to capsules having a hydrogel shell and an oil core, wherein said compound, said capsules, and water and a solvent that is miscible with both water and lipophilic compound are thoroughly mixed and incubated for a sufficient time to provide capsules loaded with a lipophilic compound. In one embodiment the ratio of the combined weight of lipophilic compound, solvent and water to the weight of capsules can be from 4:1 to 1:9, in another embodiment from 2:3 to 1:9. In one embodiment the ratio of solvent to water in the loading mixture can be from 9:1 to 1:1, in another embodiment from 1:1 to 2:1. Also provided are liquid lipophilic compound-loaded capsules prepared by a method as hereinabove described.

Without restricting the scope of the invention in any way, it is believed that the solvent that is miscible with both water and lipophile acts as a “bridge” solvent, assisting the loading of components that otherwise would have poor or no loading. Further, the resulting capsules have improved shelf stability, an increased volatile retention, and a better flowability.

The capsules that may be used in this invention are any capsules having a hydrogel shell and an oil core. Such capsules are well known to the art and are available commercially as blank capsules, ready for loading. Examples of capsules that may be used in the method of this invention are those described in U.S. Pat. No. 6,045,835 and U.S. Pat. No. 6,106,875.

The solvent can be an alcohol, such as ethanol, isopropanol and benzyl alcohol. However, other solvents, such as acetic acid, may also be used. In the case where the capsules are to be ingestible, such materials can be food grade materials. Other examples of suitable solvents include triacetin and diethyl malonate.

The mixing of the components may be achieved with the usual equipment for mixing known in the art. The components are mixed until evenly distributed; this will depend on the components used and on the order the components are added, and will take typically about 15-30 minutes. Components of the mixture can be added in any order. A suitable order to minimise mixing time is as follows: (1) Add dry capsules, water and alcohol and mix about 5-15 minutes, (2) add lipophile and mix for about 2-7 minutes.

The time to load the lipophile will vary with the natures of the lipophile, the capsules and the alcohol-water mixture, but it typically lies between about 1 and about 48 hours, about 12- about 30 hours usually being sufficient for most compounds.

The process of the invention may be used to load any suitable lipophile to a capsule. Examples of such lipophiles include (but are not limited to) flavors, fragrances, pharmaceuticals, and the like. The method allows the ready encapsulation of such substances. Without wishing to be bound by any explanation, it is believed that the system provided allows better penetration of the hydrogel, leading to better encapsulation and less plating on or trapping in capsule walls. The use of the terms “lipophile” and “lipophilic compound” encompasses the possibility that more than one such compound may be present.

Also provided are lipophile-loaded capsules prepared by a method as hereinabove described.

Also provided is a process of incorporating a lipophile into a product, comprising the addition of the lipophile in the form of an encapsulated product as hereinabove described.

Further provided is a product comprising a lipophile, the lipophile being encapsulated in a capsule as hereinabove defined.

The method provided can be used for the loading of flavours and fragrances. Also provided is a process of flavoring or fragrancing a product, comprising the addition thereto of flavor- or fragrance-loaded capsules as hereinabove described.

Further provided is a flavored or fragranced product, comprising flavor- or fragrance-loaded capsules as hereinabove described.

The flavoured or fragranced product may be any product that requires the incorporation of a flavor or a fragrance. Non-limiting examples of such products include foodstuffs and beverages of all kinds, confectionery, tobacco products, soaps, detergents and washing materials, mouthwashes and dentifrices, and the like.

The method is further illustrated by reference to the following example, which describes an embodiment and which should not be construed as limiting the invention in any way.

### EXAMPLE

### Ethanol-Mediated Loading of Capsules with Limonene

Dry crosslinked capsules (750 g), deionized water (50.0 g), and ethanol (50.0 g) are placed in a stainless steel mixing bowl (Hobart Lab Scale Mixer) and mixed for 15 minutes. A lipophilic flavor compound, limonene (150.0 g), is then added to the hydrated capsules, and mixing is continued at power level 1-2 for 5 min.

The mixture is transferred into a storage container and the container is closed. The mixture is allowed to incubate for 24 h for flavor loading before the product is used.

It will be understood that the embodiment(s) described herein is/are merely exemplary, and that one skilled in the art may make variations and modifications without departing from the spirit and scope of the invention. All such variations and modifications are intended to be included within the scope of the invention as described hereinabove. Further, all embodiments disclosed are not necessarily in the alternative, as various embodiments of the invention may be combined to provide the desired result.

1. A method of loading a liquid lipophilic compound to capsules having a hydrogel shell and an oil core, wherein said lipophilic compound, said capsules, and water and a solvent that is miscible with both water and the lipophilic compound are thoroughly mixed and incubated for a sufficient time to provide capsules loaded with the lipophilic compound.

2. The method according to claim 1, in which the ratio of the combined weight of lipophilic compound, solvent and water to the weight of capsules is from about 4:1 to about 1:9, optionally from about 2:3 to about 1:9

3. The method according to claim 1, in which the ratio of solvent to water in the loading mixture is from about 9:1 to about 1:1, optionally from about 1:1 to about 2:1.

4. The method according to claim 1, in which the solvent is an alcohol, optionally an alcohol selected from the group consisting of ethanol, isopropanol and benzyl alcohol.

5. The method according to claim 1, in which the lipophilic compound is a fragrance or a flavour.

6. Lipophilic compound-loaded capsules prepared by the method according to claim 1.

7. A process of adding liquid lipophilic compound to a product, comprising the addition thereto of lipophilic compound-loaded capsules according to claim 6.

8. A product comprising a liquid lipophilic compound, the liquid lipophilic compound being provided by lipophilic compound-containing capsules according to claim 6.

9. The product of claim 8 wherein the lipophilic compound is a flavour or fragrance.

10. The process of claim 7 wherein the lipophilic compound is a flavour or fragrance.

11. The lipophilic compound loaded capsules of claim 6 wherein the capsules, lipophilic compound and solvent are food grade materials.

12. The method of claim 1 wherein the solvent is acetic acid.

13. The method of claim 1 wherein the solvent is at least one of triacetin or diethyl malonate.

14. The method of claim 1 wherein the solvent is benzyl alcohol.
